(19) United States
(12) Patent Application Publication
(10) Pub. No.: US 2024/0255500 A1
(43) Pub. Date: Aug. 1, 2024

(54) SYSTEM AND METHOD FOR POINT OF NEED DIAGNOSTICS

(71) Applicant: Illinois State University, Normal, IL (US)

(72) Inventors: Jun-Hyun Kim, Bloomington, IL (US); Jeremy D. Driskell, Bloomington, IL (US)

(21) Appl. No.: 18/018,968

(22) PCT Filed: Jul. 29, 2021
(86) PCT No.: PCT/US21/43653

§ 371 Date: Jan. 31, 2023

Related U.S. Application Data
(60) Provisional application No. 63/059,688, filed on Jul. 31, 2020.

(51) Int. Cl. G01N 33/543; G01N 21/65

(52) U.S. Cl. CPC G01N 33/54388; G01N 21/658

(57) ABSTRACT

A diagnostic method that includes providing a plasmonic paper and an absorbing pad positioned under the plasmonic paper, pre-immobilizing an antibody onto the plasmonic paper, introducing a sample solution to the plasmonic paper to extract and concentrate antigen in the sample on the plasmonic paper, absorbing the remainder of the sample solution with the absorbing pad, passing Extrinsic Raman Labels through the plasmonic paper to label captured antigens, and detecting captured antigen.

### CROSS-REFERENCE TO RELATED APPLICATION

The present disclosure is a U.S. National Phase Entry of International Application No. PCT/US21/43653 and claims the benefit of U.S. Provisional Application No. 63/059,688 filed Jul. 31, 2020, the contents of which are hereby incorporated herein in entirety.

### TECHNICAL FIELD

The present teachings are related to a point of need diagnostic system and method and more particularly to a point of need diagnostic system and method that utilizes rapid vertical flow.

### BACKGROUND

Point-of-need (“PON”) tests are essential for early diagnosis of disease and identification of biological threats. These assays must be physically and operationally portable to work in many settings including medical offices, in home, or in the field for military and environmental applications. In practice, these applications necessitate that the assay is easy to perform, rapid, and cost-effective, while sensitivity, precision, and quantitation are desirable. Currently, the lateral flow assay (“LFA”) format has found the most success as point-of-need tests because this format provides the requisite qualities of convenience, speed, and low-cost. However, conventional LFAs that rely on colorimetric readout do not provide low detection limits that often translate into a high rate of false negative tests. Moreover, these LFAs are not quantitative and do not allow for multiplexed detection. With these limitations, much effort has been placed on improving readout strategies to improve LFA sensitivities and moderate success has been reported for detection schemes using enzymes, chemiluminescence, fluorescence, and surface-enhanced Raman spectroscopy (“SERS”). Despite efforts to develop advanced readout technologies for LFAs, this format is inherently limited by the hook effect, which complicates quantitative analysis and results in false negative results at high concentrations.

Recently, rapid vertical flow (“RVF”) assays have been explored as alternatives to LFAs that circumvent the inherent limitations of LFAs. The sequential application and immuno-reaction of the sample and label on the capture substrate eliminates the potential for the hook effect and results are immediately available without the need for assay development time. However, RVF assays that rely on visual readout also lack sensitivity and clinical accuracy. Thus, RVF assays must be coupled with emerging detection technologies to fully address the limitations of current PON tests. Of the detection modalities already explored for LFA-based PON assays, SERS offers potential advantages with respect to sensitivity and multiplexed detection capabilities.

In these SERS-PON works, conventional protein-binding membranes were employed as the capture substrate and Raman reporter functionalized nanoparticles served as SERS tags to facilitate detection. In an attempt to garner even greater sensitivity, core-shell nanoparticles have been developed to improve SERS enhancement relative to previously utilized spherical gold nanoparticles as the core of the SERS tag. However, plasmonic coupling between nanoparticles affords the greatest SERS enhancement; thus, the use of novel plasmonic particles alone does not realize the full potential of SERS in PON tests. One example utilizes a gold-plated membrane filter as a capture substrate for use in a SERS-based flow-through immunoassay that supported plasmonic coupling with SERS tags. However, those membrane filters were not amenable to the VFA format in which sample is passed through the filter by capillary action using an absorbent pad and a syringe can be used to pass sample/labeling solution through the filter thereby limiting ease of use and suffering from variable flow rates that marginalized precision. Also developed is a SERS-based PON test based on antigen-mediated aggregation of nanoparticle SERS tags in solution followed by capture and concentration of the aggregates using a RVF device to take advantage of plasmonic coupling to generate large signal enhancements. However, the aggregation-based strategy required 60 min to perform and suffered from the hook effect.

With the current efforts to develop paper-based plasmonic substrates, there exists an opportunity to significantly improve SERS-based RVF assay.

### SUMMARY

One embodiment of this disclosure is a diagnostic method that includes providing a plasmonic paper and an absorbing pad positioned under the plasmonic paper, pre-immobilizing an antibody onto the plasmonic paper, introducing a sample solution to the plasmonic paper to extract and concentrate antigen in the sample on the plasmonic paper, absorbing the remainder of the sample solution with the absorbing pad, passing Extrinsic Raman Labels through the plasmonic paper to label captured antigens, and detecting captured antigen.

In one example of this embodiment, the plasmonic paper is AuNP-loaded. In another example, the plasmonic paper is formed from grade 4 or 40 Whatman filter paper with nominal pore sizes of about 25 μm or 8 μm, respectively. In yet another example, the plasmonic paper is immersed in an AuNP suspension for about 24 hours before being removed and used in the diagnostic method. In another example, the pre-immobilizing the antibody step utilizes about 2 μg of antibody. In yet another example, about 200 μL of Extrinsic Raman Labels are passed through the plasmonic paper treated with about 100 ng/ml antigens. In yet another example, the detecting step utilizes visual images of the plasmonic paper.

Yet another example of this embodiment includes adding an additional layer of structurally diverse plasmonic nanoparticles. In part of this example, the additional layer comprises any one or more gold-based nanoparticles having a sphere gold, an anisotropic gold, or concave cubic gold structure.

Another example of this embodiment includes introducing an additional layer with plasmonic nanoparticles onto the plasmonic paper.

Yet another embodiment of this disclosure is a diagnostic method that includes providing a plasmonic paper and an absorbing pad positioned under the plasmonic paper, pre-immobilizing an antigen onto the plasmonic paper, introducing a sample solution to the plasmonic paper to extract and concentrate antibodies in the sample on the plasmonic paper, absorbing the remainder of the sample solution with the absorbing pad, passing Extrinsic Raman Labels through the plasmonic paper to label captured antibodies, and detecting captured antibodies.

In one example of this embodiment, the plasmonic paper is AuNP-loaded. In another example, the plasmonic paper is formed from grade 4 or 40 Whatman filter paper with nominal pore sizes of about 25 μm or 8 μm, respectively. In yet another example, the plasmonic paper is immersed in an AuNP suspension for about 24 hours before being removed and used in the diagnostic method. In another example, the pre-immobilizing the antibody step utilizes about 2 μg of antibody. In yet another example, about 200 μL of Extrinsic Raman Labels are passed through the plasmonic paper treated with about 100 ng/ml antigens. In another example, the detecting step utilizes visual images of the plasmonic paper.

Another example of this embodiment includes adding an additional layer of structurally diverse plasmonic nanoparticles. In part of this example, the additional layer comprises any one or more gold-based nanoparticles having a sphere gold, an anisotropic gold, or concave cubic gold structure.

Another example of this embodiment includes introducing an additional layer with plasmonic nanoparticles onto the plasmonic paper.

### DESCRIPTION OF THE DRAWINGS

The above-mentioned aspects of the present disclosure and the manner of obtaining them will become more apparent and the disclosure itself will be better understood by reference to the following description of the embodiments of the disclosure, taken in conjunction with the accompanying drawings, wherein:

FIG. 1a is one embodiment of a vertical flow immunoassay configuration;

FIG. 1b is one embodiment of a vertical flow immunoassay protocol;

FIG. 2 illustrates SEM images of filter paper with corresponding UV analysis;

FIG. 3 illustrates surface UV-Vis-IR spectra of AuNPs loaded onto grade number 4 and 40 filter papers;

FIGS. 4a-4c illustrate graphical data wherein ERLs were passed through the capture substrates and analyzed via SERS;

FIG. 5 illustrates graphical data of a SERS spectra collected using bare filter paper, plasmonic grade 4 filter paper, and grade 40 plasmonic filter paper as the capture substrate in a SERS-RVF immunoassay;

FIGS. 6a-6c illustrate graphical data of SERS assays with optimization parameters;

FIG. 7a illustrates graphical data of concentration-dependent SERS spectra;

FIG. 7b illustrates graphical data of a single band observed and plotted as a function of antigen concentration to generate a calibration curve;

FIGS. 8a-8b illustrate graphical data of calibration curves for two independent assays;

FIG. 9a illustrates graphical data for results of SERS spectra intensity obtained using the optimized conditions;

FIG. 9b illustrates graphical data for a calibration plot of the VFA obtained using the optimized conditions with limit of detection.

FIG. 10 illustrates graphical data for one embodiment of calibration curves generated with different solvents;

FIGS. 11a-11c illustrate a scheme for formation of a third layer onto VFA paper with structurally diverse plasmonic nanoparticles;

FIG. 11d illustrates graphical data that representative SERS signals obtained after applying spherical, anisotropic, and cubic AuNPs onto VFA paper treated with 100 ng/ml mouse IgG; and

FIG. 12 illustrates one embodiment of a diagnostic method implementing the teachings of the present disclosure.

Corresponding reference numerals are used to indicate corresponding parts throughout the several views.

### DETAILED DESCRIPTION

The above-mentioned aspects of the present application and the manner of obtaining them will become more apparent and the teachings of the present application itself will be better understood by reference to the following description of the embodiments of the present application taken in conjunction with the accompanying drawings.

The present disclosure relates to cost-effect, robust, and reproducible system and method to easily fabricate plasmonic papers that are ideal for incorporating into a surface-enhanced Raman spectroscopy (“SERS”) rapid vertical flow (“RVF”) assay as the capture substrate. This technology is discussed in the publication entitled “Rapid vertical flow immunoassay on AuNP plasmonic paper for SERS-based point of need diagnostics” and authored by Frimpong R, Jang W, Kim J H, Driskell J D, the contents of which are hereby incorporated herein by reference in entirety. Embedded gold nanoparticles in the filter paper create small gaps with the bound SERS nanoparticle tag that forms a SERS hot spot to significantly enhance the signal produced by the SERS tag compared to the same SERS tag bound to a non-plasmonic support. In one example of this disclosure, a layered gold nanoparticle system can be used to model a SERS RVF configuration and established marked improvement in SERS enhancement provided by the underlying plasmonic support. Moreover, the pore sizes of the filter paper can be selected to optimize flow speed to maximize antibody-antigen binding efficiency and minimize assay time and nanoparticle loading to maximize coupling and SERS enhancement with the binding of SERS tags bound to captured antigen.

In one aspect of this disclosure, AuNP embedded filter paper may be used as a capture substrate in a SERS-based RVF assay as a highly sensitive, rapid, and easy to use point of need (“PON”) test that affords quantitative accuracy. Further, this disclosure is easily adaptable for the detection of a variety of target antigens using appropriate antibodies. The plasmonic substrate may be demonstrated to provide a significant increase in assay sensitivity. After establishing the role of the plasmonic capture substrate, the analytical performance of the optimized assay may be assessed based on the detection limit, sensitivity, specificity, and precision among other things. The concentration of immunoglobulin G (“IgG”) in normal mouse serum may be accurately quantified using the developed SERS RVF assay to demonstrate potential for the analysis of clinical samples among other things. Further, the present disclosure refers specifically to a model system that was tested to establish proof of concept. A person skilled in the art understands that the present disclosure is adaptable for the detection of a variety of antigens (or antibodies).

### Reagents and Materials

Gold nanoparticles (“AuNPs”) may be used to synthesis extrinsic Raman labels (“ERLs”). In one aspect of this disclosure, the AuNPs are about 60 nm. However, AuNPs larger and smaller than 60 nm are also considered herein. Other reagents and materials considered for this disclosure include Bovine serum albumin (“BSA”), 4-nitrobenzenethiol (“4-NBT”), Tween 20, mouse IgG (“15381”), goat anti-mouse IgG polyclonal antibody (“ab 7063”), normal mouse serum, phosphate buffered saline (PBS; 10 mM, pH 7.4), and sodium borate buffer (50 mM, pH 8.5) among others. In one non-exclusive example, Whatman filter paper grades 4 and 40 may be used to prepare SERS substrate. This disclosure also contemplates the use of a vertical flow device (i.e, a Zoom Blot Plate) and high capacity absorbing pads.

### Synthesis of Plasmonic Nanoparticles

In one aspect of this disclosure, structurally diverse plasmonic nanoparticles (sphere, cube, anisotropic, rod, etc.) may be synthesized and subsequently used to prepare plasmonic paper. For example, spherical AuNPs with ˜60 nm diameters may be synthesized using a modified thermal reduction method. An aliquot (2.0 mL) of gold (1 wt % HAuCl₄·3H₂O) solution may be diluted to about 100 mL with water in a 250 mL Erlenmeyer flask containing a magnetic stirring bar. After vigorously stirring the solution for about 15 minutes, the flask may be heated to boiling. Trisodium citrate (1 wt %; 1.5 mL) may be quickly added to the boiling solution, which leads to the formation of spherical AuNPs. After completing the reaction, the final solution may be adjusted to about 80 mL in total volume (exhibiting an extinction maximum of ˜2.4) and stored at room temperature without further purification prior to use.

While one particular method for synthesis of AuNPs is specifically discussed herein with reference to corresponding volumes, times, and weights, this disclosure contemplates utilizing any other method known in the art that may produce Au-based nanoparticles suitable for the present disclosure.

### Preparation of Plasmonic Paper Capture Substrate

In one aspect of this disclosure, Whatman grade 4 and 40 filter paper with about 8 and 25 μm pores sizes, respectively, may be used to prepare the plasmonic paper capture substrate. As-prepared structurally diverse plasmonic nanoparticles may be embedded in the two filter papers. The papers may be first dried at about 50° C. in an oven until fully dried (i.e., about 12 hours) and then immersed in about 10 mL of the synthesized AuNP suspension in a plastic petri dishes (about 60 mm×15 mm). After soaking for about 24 hours, the filter papers may be removed from the AuNP suspension and at least partially dried in the oven (˜40° C.). The resulting plasmonic paper may then be cut into circles with an about 3 mm diameter for use in the SERS immunoassay. About 2 μL of 1 mg/mL goat anti-mouse antibody may be applied to the 3 mm plasmonic paper substrate and dried in a desiccator for about 1.5 hours before use.

While one particular method for preparation of plasmonic paper capture substrate is specifically discussed herein, this disclosure contemplates utilizing any other method know in the art that may prepare a plasmonic paper capture substrate suitable for the present disclosure.

### Preparation of ERLs

In one aspect of this disclosure, antibody and a Raman reporter molecule may be co-adsorbed onto AuNPs to prepare ERLs. An about 1.0 mL aliquot of about 60 nm AuNPs may be added to a microcentrifuge tube and about 40 μL of about 50 mM borate buffer (about pH 8.5) may be introduced to adjust the pH. Goat anti-mouse IgG antibody (about 30 μg) and about 10 μL of about 1 mM 4-NBT may be added and incubated for about 1 hour to allow the antibody and 4-NBT to adsorb onto the AuNPs, forming the ERLs. The ERL suspension may be centrifuged at about 5000 g for about 5 min and the supernatant above the pelleted ERLs may be discarded to remove excess antibody and 4-NBT that was not adsorbed to the AuNPs. The ERLs may be resuspended in about 2 mM borate buffer and the centrifugation/resuspension cycle may be repeated two additional times. To the purified ERLs, about 10 μL of 10% BSA may be added to further passivate any remaining surface on the AuNPs and about 10 μL of 10% NaCl may be added to mimic physiological ionic strength and prevent protein unfolding.

While one particular method for preparing ERLs is specifically discussed herein, this disclosure contemplates utilizing any other method know in the art that may prepare ERLs suitable for the present disclosure.

### Vertical Flow Immunoassay Protocol

Calibration standards of the antigen may be prepared by diluting about a 1 mg/ml stock solution of mouse IgG to final concentrations of about 0.1, 0.5, 1, 10, 50, 100, 200, 300 and 400 ng/mL. Two sets of calibration standards may be prepared, one using phosphate buffer saline (“PBS”) as the diluent and another using rabbit serum as the diluent. PBS or rabbit serum may serve as the negative control to assess nonspecific binding and specificity. The capture substrate may be comprised of the plasmonic filter paper with pre-adsorbed goat anti-mouse IgG antibody and loaded on the adsorbing pad and inserted into a vertical flow device.

Once assembled, about 1% BSA (about 100 μL) can be added to the sample well, and rapidly passed through the capture substrate into the absorbing pad to block the capture substrate and sample well surface in an effort to prevent nonspecific binding. The sample solution, e.g., calibration standard or normal mouse serum, may be added to the sample well and antigen molecules may be captured on the plasmonic paper by the immobilized antibody as the solution freely passes through the capture substrate. Next, ERLs may be added to the sample well and pass through the capture substrate to label any captured antigen, with excess ERLs drawn into the absorbing pad. Excess ERLs may be removed from the surface of the plasmonic paper by passing a PBS rinse solution containing about 5% Tween 20 through the paper. The plasmonic paper may be removed and allowed to dry in a desiccator before SERS analysis.

While one particular vertical flow immunoassay protocol is specifically discussed herein, this disclosure contemplates utilizing any other method know in the art suitable for the present disclosure.

### Instrumentation

In one aspect of this disclosure, the plasmonic papers may be examined using an FEI-Quanta 450 SEM operating at 20 kV or any known instrument capable of producing visual images of the papers. Prior to this analysis, the papers may have been coated with a thin gold film using a Denton vacuum sputter coater (DESK II) or any other similar method, to avoid common charging problems. The images may be collected to measure the general size distribution of plasmonic particles and to visualize overall packing patterns of the NPs on the surface of the filter paper among other things.

To evaluate SERS response of the vertical flow assay, SERS spectra may be collected using an Enwave Optronics, Inc. ProRaman-L-785B instrument configured with a 785 nm excitation source set to 10 mW at the sample surface and a high-sensitivity CCD thermoelectrically cooled to −60° C. or any other known instrument and method. The sample may be placed on an x-y-z sample stage and the laser focused on the substrate surface by maximizing the SERS intensity. After focusing, the sample stage may be automated to move linearly along the x-direction during the 10-s spectral acquisition as a means of increasing the sampling area on the capture substrate, effectively averaging signal from different locations on the substrate. Five spectra may be collected from each sample substrate and baseline corrected using an auto-baseline-2 algorithm or any other known method.

The adsorption of the antibodies on the AuNPs of the ERL may be confirmed by measuring the mean hydrodynamic diameter and polydispersity of the AuNPs before and after the preparation of the ERLs. In one non-exclusive example, this is done with a Malvern Zetasizer Nano ZSP. However, any known instrument capable of this measurement is considered herein. Unconjugated AuNP and synthesized ERLS may be diluted about 2-fold with nano pure water and placed in a micro-volume disposable Eppendorf cuvette for DLS analysis. The sample may be equilibrated for about 60 seconds prior to analysis and each size measurement may be determined from 10 runs, 10 seconds each.

The synthesized gold nanoparticles used to prepare the plasmonic paper may be investigated to assess the number of AuNPs loaded on the filter paper by measuring the extinction of the AuNP suspension before and after adsorption onto the filter paper. In one non-exclusive example, the UV-visible instrument used for this analysis may be the Agilent 8453 spectrophotometer (Agilent Technologies, Santa Clara, CA). The instrument has photodiode detector with a spectral range of 190-1100 nm. Prior to sample analysis the instrument may be blanked with about 2 mM borate buffer (pH 8.5). The prepared plasmonic paper may be characterized using a surface UV-Vis-IR spectrophotometer (300-1700 nm), among others, equipped with a reflectance probe (StellarNet).

While particular instruments are specifically discussed herein, this disclosure contemplates utilizing any other instruments known in the art that may be suitable for the present disclosure.

### Assay Design and Sensing Principle

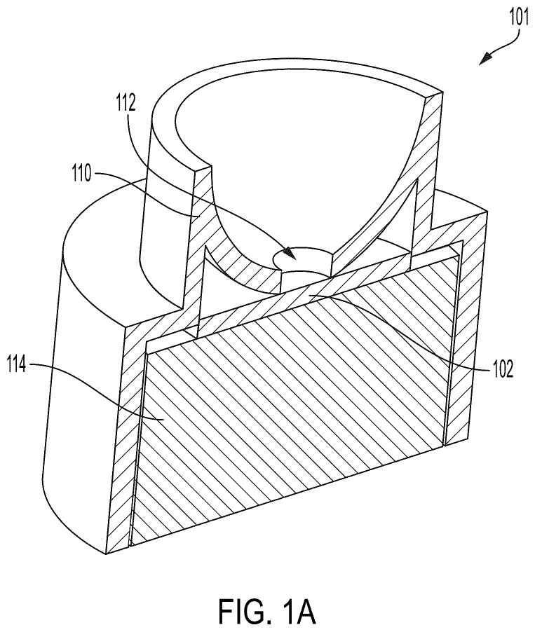

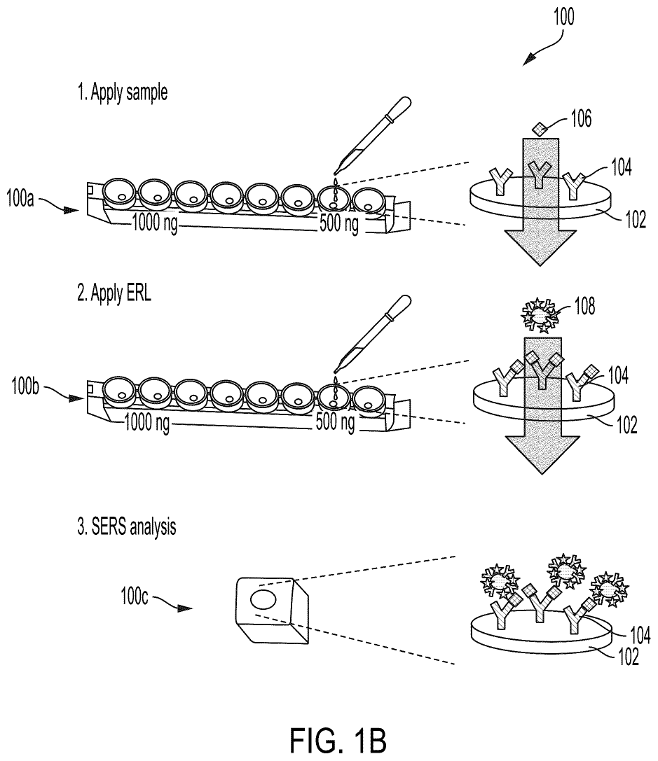

Referring now to FIGS. 1a-1b, one embodiment of a vertical flow immunoassay configuration 101 and protocol 100 is illustrated. The vertical flow immunoassay configuration 101 may include one or more vertical flow well 110 configured to direct a sample solution 106 introduced into the vertical flow well 110 to a through-hole 112. Adjacent to the through-hole 112 may be a plasmonic filter paper 102 that has been prepared as discussed in this disclosure. On the side of the plasmonic filter paper 102 opposite the through-hole 112 may be an absorbing pad 114. The absorbing pad 114 may be a material capable of absorbing any sample that flows through, but is not absorbed by, the plasmonic filter paper 102.

In a first step 100a, the sample solution 106 may be vertically passed through the plasmonic filter paper 102 for antibody 104 to extract the analyte. In the second step 100b, ERLs 108 are passed through the plasmonic filter paper 102 to label the captured antigen. In a third step 100c, a SERS analysis may be performed wherein a photograph of the assembled vertical flow device and the disassembled apparatus with a capture substrate placed on the absorption pad is analyzed.

In the embodiment of FIGS. 1a-1b, the plasmonic filter paper 102 may be AuNP-loaded plasmonic paper, which serves as a capture substrate for the detection of antigens. Antibody 104 is pre-immobilized onto the plasmonic filter paper 102 and antigen is extracted and concentrated on the substrate as the sample solution 106 passes through to the absorbing pad 114 via capillary action. In the second step 100b, ERLs 108 are spontaneously and vertically passed through the plasmonic filter paper 102 to specifically label captured antigen 108 and facilitate SERS-based detection. The micron-sized pores in the plasmonic filter paper 102 allow any excess nanometer-sized ERLs to rapidly and freely pass through the plasmonic filter paper 102 to waste collected in the absorbing pad 114.

The plasmonic filter paper 102 provides at least two features. First, antibody irreversibly 104 adsorbs onto AuNPs embedded in the plasmonic filter paper 102, to form a robust capture substrate that resists desorption and loss of function while solutions flow through the plasmonic filter paper 102. Second, the AuNP embedded in the plasmonic filter paper 102 will form a sandwich-like structure with ERLs bound to captured antigen. This architecture greatly supports plasmonic coupling between the AuNPs to generate a large localized electric field between the sandwiched nanoparticles and significantly enhance the SERS signal relative to the isolated ERL in the absence of plasmonic coupling. In addition to the uniquely designed plasmonic paper to maximize analytical signal, the vertical flow format overcomes diffusional mass transport limitations of traditional immunoassays to substantially reduce assay time and does not suffer from the hook effect, as is the case for lateral flow assays, to improve quantitative capabilities.

In one aspect of this disclosure, the plasmonic capture substrate and ERLs were both synthesized with goat anti-mouse IgG polyclonal antibody and mouse IgG was employed as the antigen. The following sections detail the synthesis of the plasmonic paper, the role of the plasmonic paper to facilitate large SERS enhancements, and the assay parameters that impact analytical performance. After optimization, the teachings of this disclosure can define the analytical figures of merit and apply the protocol to accurately quantify IgG in normal mouse serum among other things.

### Fabrication and Characterization of Plasmonic Paper

Plasmonic filter paper 102 loaded with spherical AuNPs may serve as the capture substrate in the SERS-based vertical flow assay illustrated in FIGS. 1a-1b. The plasmonic filter paper 102 of this disclosure may be selected because of its affordability, accessibility, biodegradability, disposability, modification, and variety of unique pore sizes among other things. Further, other types of filter paper are also contemplated herein. In one non-exclusive example of this disclosure, the plasmonic filter paper 102 may be prepared with Grade 4 and 40 Whatman filter paper with nominal pore sizes of about 25 μm and 8 μm, respectively.

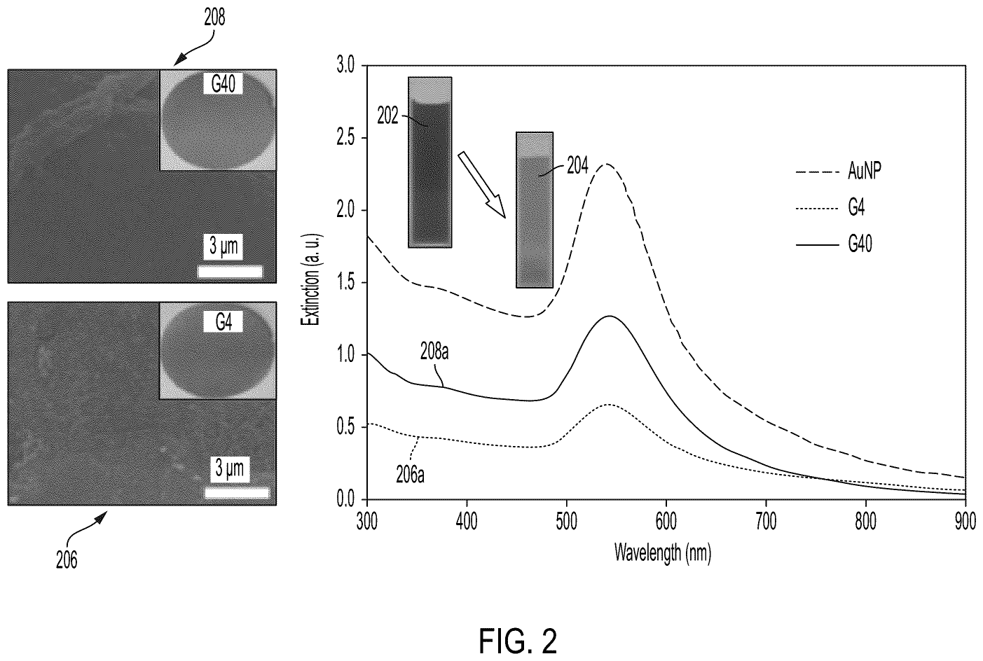

FIG. 2 shows digital photographs of the two filter papers 202, 204 before and after loading with AuNPs, prepared by dip coating. Further, FIG. 2 illustrates SEM images of filter paper grade number 4 (see 206) and 40 (see 208) with their corresponding UV analysis 206a, 208a of AuNPs solution before and after loading on the papers.

The change in color of the filter paper from white to purple indicates the adsorption of the AuNPs on the paper, and the consistent color across the full paper suggests uniform distribution on a macroscopic scale. SEM images may be used to confirm adsorption of the AuNPs onto the fibers of the filter paper 202, 204 with a mean diameter of about 57.9±12.5 nm. The images may further reveal small aggregates consisting of a few AuNPs formed on the filter paper 202, 204 during the dip coating process. Moreover, the SEM images may show a greater loading density of AuNPs on the grade 4 filter paper 206 than the grade 40 filter paper 208. The AuNP adsorption efficiency may be quantified by the changes of AuNP surface plasmon resonance (“SPR”) using UV-visible spectrophotometry or any other known method.

In one non-exclusive example, the AuNP suspension exhibited an extinction of 2.4 at 540 nm before filter paper was dipped into the suspension. After immersing the filter paper in the AuNP suspension for about 24 hours and subsequent removal, the remaining suspension displayed an extinction of about 0.7 and 1.3 for the grade 4 and 40 filter papers, respectively (See FIG. 2). These decreases in SPR bands represent the loading of 2.8×10¹¹ AuNPs and 1.8×10¹¹ AuNPs on the grade 4 and 40 filter papers, respectively.

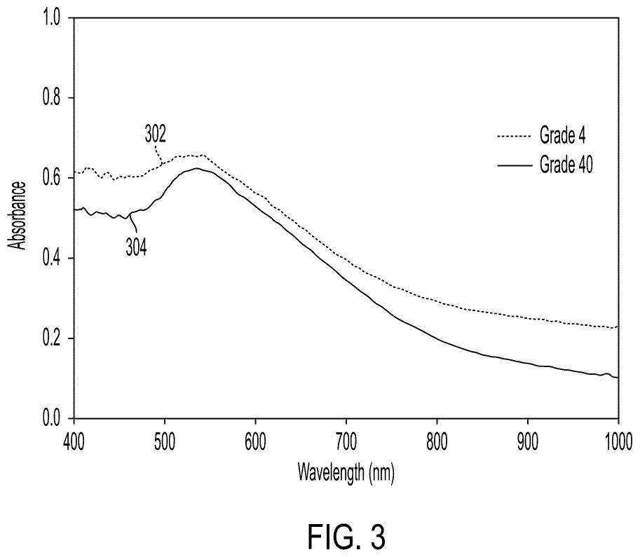

FIG. 3 illustrates Surface UV-Vis-IR spectra of AuNPs loaded onto grade number 4 (see 302) and 40 (See 304) filter papers. To support the detectably different amounts of plasmonic AuNP loading onto these two papers, the surface UV-Vis-IR absorption patterns may be obtained as illustrated in FIG. 3. Although obtaining surface absorption patterns by a reflectance probe may be difficult, the clearly different SPR patterns (i.e., broader SPR band at 545 nm and increased background peak over 700 nm) may indicate that the grade 4 filter paper possessed a relatively higher number of locally aggregated AuNPs than the grade 40 filter paper. The presence of slightly more and dense packing of AuNPs could induce a higher probability of effective plasmonic coupling that can favorably influence the degree of SERS enhancements.

### Role of Plasmonic Paper

SERS-based vertical and lateral flow immunoassays may use paper and nitrocellulose membranes as capture substrates. One aspect of this disclosure is to capitalize on the benefit afforded by a plasmonic capture substrate to create sandwich-like structures and achieve greater signal enhancements. To establish the value of the plasmonic capture substrate, the vertical flow assay may be performed using unmodified filter paper and plasmonic filter paper. Antibody may be deposited on unmodified filter paper and AuNP-embedded filter paper to form the capture substrate. After blocking the paper with BSA, 100 μL of the positive control sample (100 ng/ml of mouse IgG) can be passed through the capture substrate. The sample solution quickly passed through the unmodified filter paper and grade 4 plasmonic paper in less than 10 seconds; however, the 100 μL sample required more than 2 min to flow through the grade 40 plasmonic paper. After allowing the sample to completely pass through the capture substrate to the absorbent pad, 100 μL of ERLs were precisely applied to the sample and allowed to pass through the capture substrate to label the antigen extracted by the capture substrate. Again, the ERL flow rate may be significantly faster through the grade 4 plasmonic paper than the grade 40 plasmonic paper. After rinsing the paper with PBS (100 μL), SERS spectra were collected to quantify the bound antigen. Representative SERS spectra are presented for each of the capture substrates in FIG. 3. As is evident, the SERS signal is characteristic of the Raman reporter molecule, 4-NBT, used to synthesize the ERLs, and include prominent bands at 1338 cm⁻¹ (symmetric NO₂ stretch), 1559 cm⁻¹ (C═C), 1153 cm⁻¹ (C—O), and 850 cm⁻¹ (C—H) [52, 53]. The signal is markedly increased for the plasmonic paper capture substrates relative to the capture substrate prepared with unmodified filter paper. Plasmonic paper resulted in ˜4-fold and ˜10-fold increase in signal for the capture substrate prepared with grade 40 and grade 4 filter paper, respectively, relative to the unmodified paper.

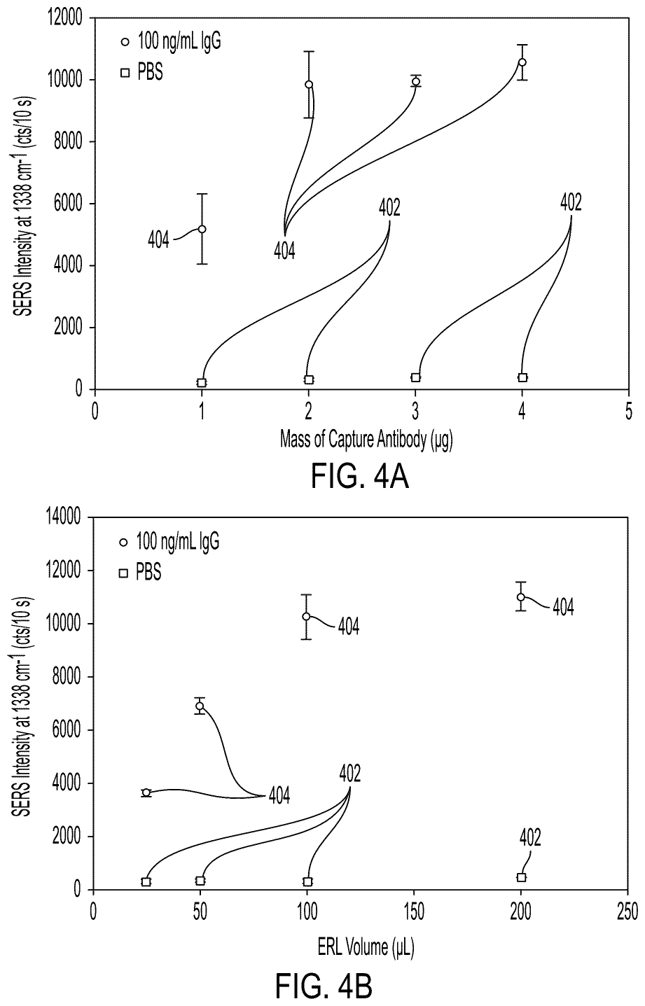

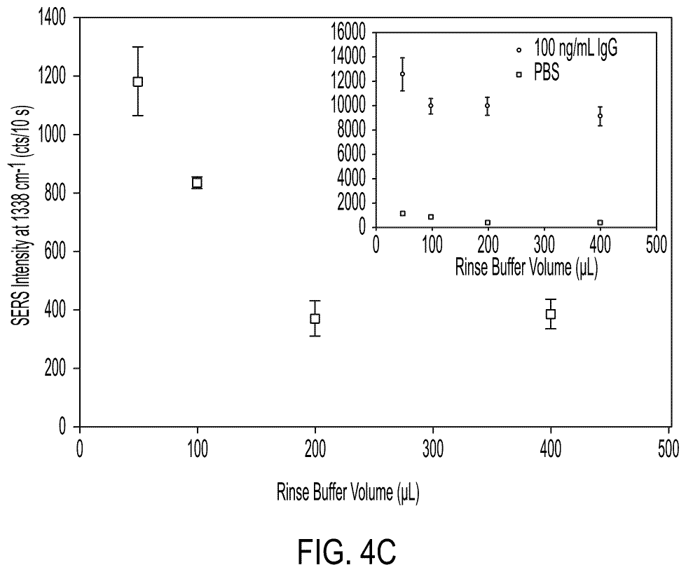

Negative control samples were also analyzed to confirm that the signals obtained for the mouse IgG sample was due to specific antibody-antigen interactions rather than non-specific binding of the ERLs (e.g., physical adsorption) in the filter paper used as the foundation of the capture substrate. To this end, PBS can be passed through the capture substrates in the first step. Subsequently, ERLs were passed through the capture substrates and analyzed via SERS (FIGS. 4a-4c). The signal measured for the PBS negative control 402 was substantially lower than that observed for the analysis of the positive control sample 404 for each of the capture substrates. These results confirm the specificity of antibody-antigen interaction yet reveal some non-specific binding of the ERLs.

Collectively, these results demonstrate greater SERS signals are obtained for the plasmonic capture substrates than the unmodified capture substrate. The plasmonic paper fabricated with grade 4 filter paper provided greater signal for the analysis of the positive control and lower signal for the analysis of the negative control sample than the grade 40 filter paper. As indicated earlier, the SERS enhancement is as a result of interparticle coupling that generates strong localized electromagnetic fields to maximize SERS signal from the substrate. Our results suggest that SERS enhancement provided by the plasmonic capture substrate corresponds to the number of plasmonic nanoparticles that are present on the surface of the paper. Moreover, the paper with the bigger pore size, e.g., grade 4, resulted in less non-specific binding of ERLs to generate less false-positive signal. Owing to this dual benefit, Whatman filter paper grade number 4 may be selected to prepare plasmonic capture substrates and optimize the assay parameters.

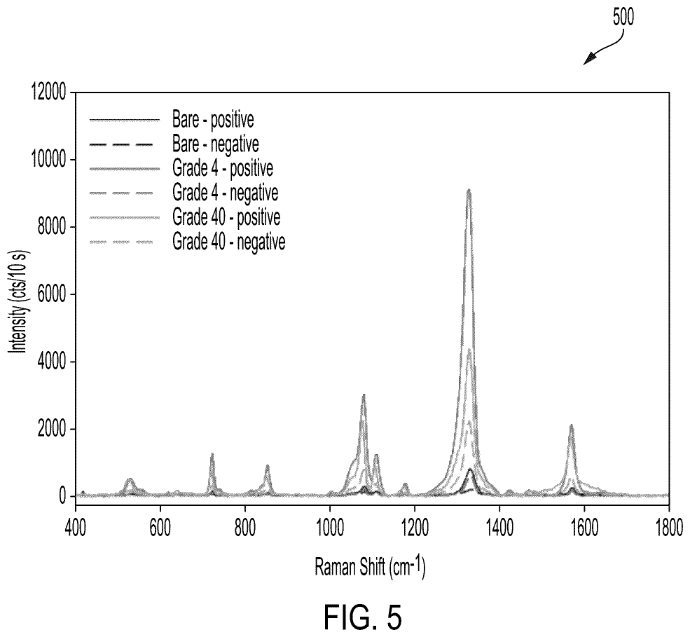

FIG. 5 illustrates a SERS spectra 500 collected using bare filter paper, plasmonic grade 4 filter paper, and grade 40 plasmonic filter paper as the capture substrate in a SERS-RVF immunoassay. The positive control samples represent the analysis of 100 ng/ml mouse IgG and the negative control samples represent the analysis of PBS.

### Assay Optimization: Amount of Capture Antibody

The ability to capture and concentrate antigen on the surface of the plasmonic paper as it passes through correlates with the number of capture antibodies available on the substrate. Therefore, the amount of the antibody deposited on the surface of the paper is a parameter that can contribute to the analytical performance of the vertical flow immunoassay. In one non-exclusive example, sufficient antibody is added to the plasmonic paper to saturate all available immobilization sites. However, addition of a large excess of antibody should be avoided to minimize the cost of the assay. To this end, goat anti-mouse IgG antibody (about 1 to 4 μg) may be deposited on plasmonic paper (grade 4) to prepare capture substrates with variable amounts of capture antibody. A positive control sample (about 100 ng/mL antigen) and negative control sample (PBS) may be analyzed with each capture substrate, using about 100 μL of ERLs and about 100 μL of PBS for subsequent labeling and rinsing, respectively. The SERS signal as a function of the amount of capture antibody is plotted for the positive and negative control samples in FIG. 4a. The positive control signal increased as the amount of deposited capture antibody increased from 1 to 2 μg; however, no further increase was observed for the positive control sample with the deposition of more than 2 μg of capture antibody. The signal for the negative control sample was independent of the mass of capture antibody immobilized on the capture substrate. Accordingly, in one non-exclusive example of this disclosure about 2 μg of capture antibody may be utilized for cost effectiveness, since this mass provided enough antibodies to effectively maximize the capture of antigen in a 100 ng/mL sample and produce a maximum SERS signal.

### Assay Optimization: Volume of ERLs

This disclosure also contemplates the effect of ERL volume on SERS intensity in an effort to maximize the analytical signal by ensuring all the captured antigen was labelled. In one non-exclusive example, plasmonic paper can be loaded with about 2 μg of capture antibody to prepare the capture substrate. A positive control sample (about 100 ng/ml antigen) or a negative control sample (PBS) can be passed through the capture substrate followed by varying volumes of ERLs (about 25, 50, 100, and 200 μL) to label the capture antigens. After rinsing with about 100 μL of buffer SERS spectra were collected, and the average intensities were plotted as a function of ERL volume in FIG. 4B. The SERS signal increases as the ERL volume increases from 25 to 100 μL, suggesting that <100 μL of ERLs is insufficient to label all of the captured antigen for a sample concentration of 100 ng/mL. While there was a slight increase in the mean signal when the ERL volume increased from 100 to 200 μL the difference was not statistically different at the 95% confidence level; thus, indicating that 100 μL of ERLs may be sufficient to label all antigens captured on the substrate for the 100 ng/mL sample. It is worth noting that analysis of samples with a concentration greater than 100 ng/ml may result in more captured antigens and therefore require a greater ERL volume to fully label all antigen molecules. The signal of the negative control samples did not statistically differ among any of the ERL volumes tested. Therefore, based on the parameters used in this disclosure, about 200 μL of ERLs may be considered.

### Assay Optimization: Volume of Rinsing Buffer

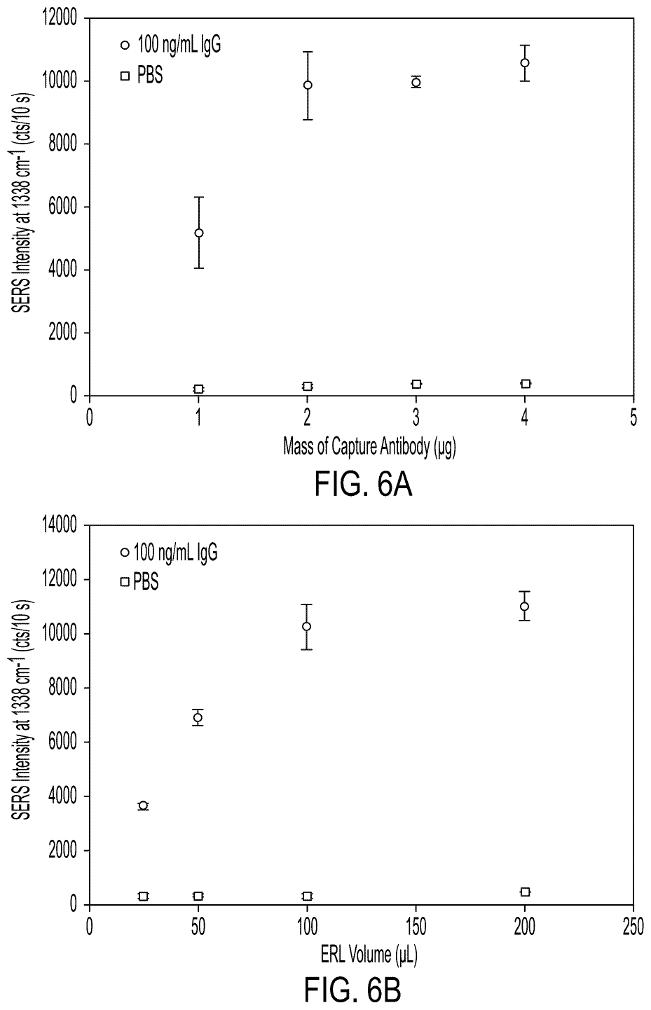

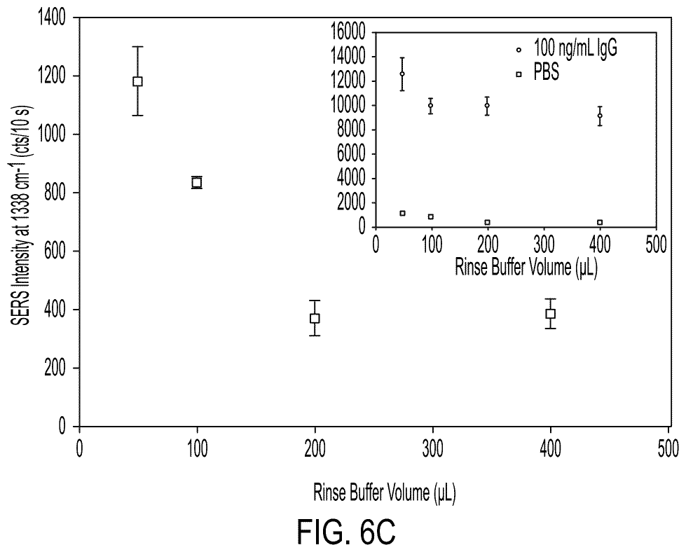

Referring now to FIGS. 6a-6c, SERS response of assay optimization parameters are illustrated. More specifically, FIG. 6a illustrates the effect of mass of goat ant-mouse IgG capture antibody deposited on the paper substrate, FIG. 6b illustrates the effect of ERL Volume, and FIG. 6c illustrates the effect of rinse buffer volume. A positive control sample (100 ng/mL mouse IgG) and a negative control sample (PBS) were analyzed for each assay condition in FIGS. 6a-6c.

One aspect of this disclosure considers the effect of rinsing volume in an effort to minimize non-specific binding. More specifically, positive (100 ng/ml antigen) and negative (PBS) were analyzed by the vertical flow assay using the optimized mass of capture antibody (2 μg) and volume of ERLs (200 μL). Following the labeling step, different volumes of the rinsing buffer were applied to the sample well and passed through the capture substrate to remove non-specifically bound ERLs. FIG. 6c plots the SERS intensities for the negative control samples as a function of rinse buffer volume. The signal for the negative control sample is a measure of the non-specific binding, and the data show that the negative control signal decreases as the volume of rinse buffer increases from 50 to 200 μL. That data indicates that excess unbound ERLs remain in the capture substrate filter paper when the rinse volume was less than about 200 μL. An increase to 400 μL of PBS rinsing buffer did not lead to a further reduction in the background signal.

FIG. 6c (inset) plots the signal collected for the positive control samples as a function of PBS rinsing volume. The data show a decrease in the signal for the positive control sample as the rinse volume increases from 50 to 100 μL, which is likely due to the removal of excess unbound ERLs. The positive control signal is independent of rinse volumes between 100 and 400 μL, which suggests that these volumes do not disrupt the specific antibody-antigen interactions. Based on the assessment of these results, this disclosure considers about 300 μL of rinsing volume for optimized assays. This volume of rinsing buffer ensures a thorough removal of excess, non-specifically bound ERLs and maintains the maximum analytical signal, while balancing the assay time. Larger volumes may require more time for the buffer to pass through the capture substrate, and it is noteworthy that the flow rate decreases as the absorbent pad becomes saturated with liquid.

### Analytical Performance—Dynamic Range, Detection Limit, Reproducibility, Sample Matrix

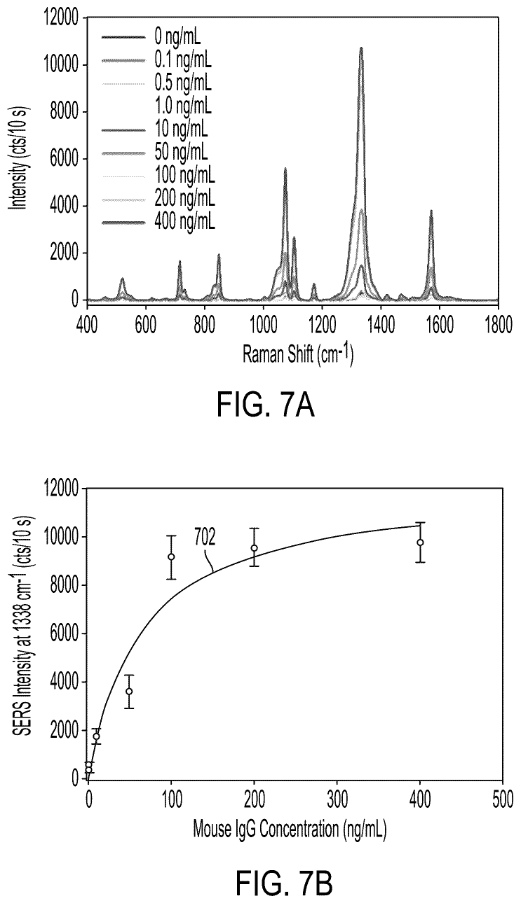

In one aspect of this disclosure, the analytical performance of the assay can be evaluated using the optimized conditions to generate a calibration curve for the analysis of mouse IgG and establish the dynamic range, detection limit, and reproducibility among other things. Three assays for mouse IgG were performed on three different weeks using three independently prepared suspensions of ERLs and three independently prepared sets of plasmonic paper. A calibration curve can be generated for each independent assay to allow for analysis of intra- and inter-assay reproducibility. Concentration-dependent SERS spectra are presented in FIG. 7a for one calibration dataset. As is evident, the SERS intensity increases with a corresponding increase in antigen concentration. As one non-exclusive example, the most intense band observed at 1338 cm⁻¹, due to the symmetric nitro stretch, is plotted as a function of antigen concentration to generate a calibration curve 702 (see FIG. 7b).

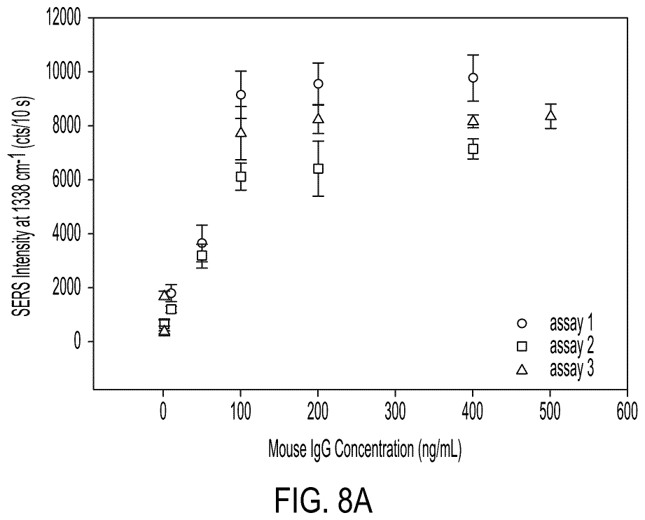

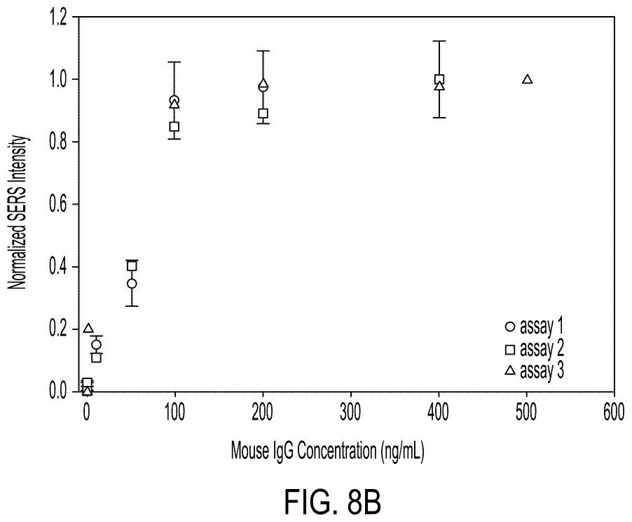

Each data point represents the average signal collected from five different locations across the capture substrate and the error bars represent the standard deviation. The spot-to-spot variation in analytical signal measured 10-20% for most samples, although at low concentrations, e.g., 0.5 ng/mL, where the signal was small, it varied as much as 37%. Calibration curves for two additional independent assays are presented in FIGS. 8a and 8b. Analysis of the raw signals to evaluate the inter-assay reveals that the percent relative standard deviations (% RSD) are 10-30% for a given concentration. Closer inspection of the calibration curves shows that each dataset has a similar curve shape, in which the signal increases for sample concentrations between 0 and 100 ng/ml before reaching a maximum signal as the capture substrate becomes saturated at concentrations greater than 100 ng/mL. Each of the three independent assays shows a slightly different maximum signal at saturation. While the dynamic range and overall curve shape, i.e., binding behavior, is similar for each assay, the differences in maximum signal are attributed to differences in the synthesized plasmonic paper substrates responsible for the SERS enhancement. To account for the differences in plasmonic paper enhancement, each calibration curve can be normalized with respect to its maximum signal (See FIG. 8b). After normalization to account for substrate differences the inter-assay variability was significantly reduced to ˜10-20%.

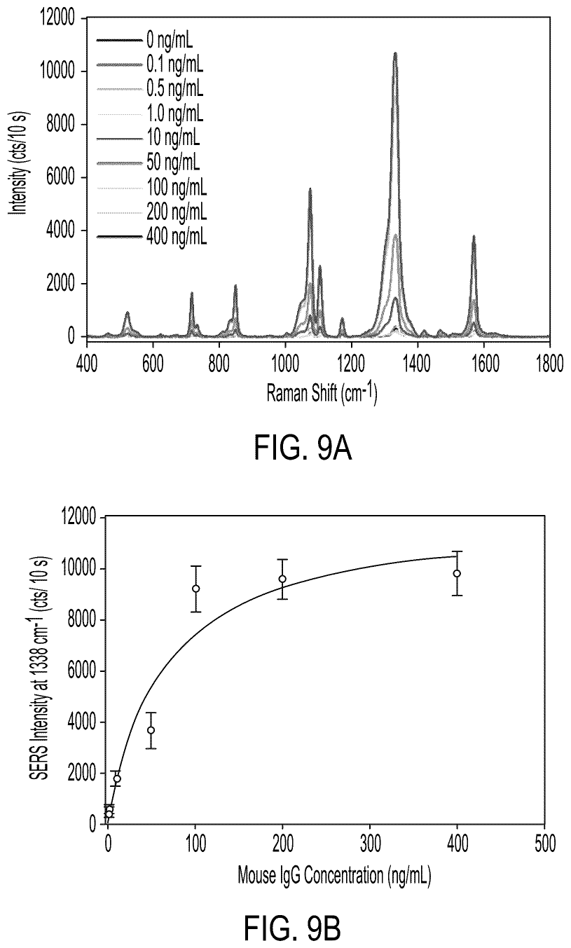

FIGS. 9a-9b illustrate results of SERS spectra intensity obtained using the optimized conditions, optimized conditions with varied mouse IgG concentrations in ng/ml, comparison of calibration plot between filter paper grade number 4 AuNP-plasmonic and bare filter paper obtained using optimized conditions, and a calibration plot of the VFA obtained using the optimized conditions with limit of detection.

The detection limit of the SERS vertical flow assay can be defined as the antigen concentration that produces a signal equal to the blank signal plus three times the standard deviation of the blank measurement. The calibration data presented in FIG. 9b can be best fit to a ligand binding model (i.e., saturation curve-See Equation 1):

$$y = \frac{{B}_{m ax} x}{{K}_{d} + x} \qquad Equation 1$$

where Bₘₐₓ represents the maximum binding (i.e., maximum signal), and K_{d} is the antibody-antigen dissociation constant. Based on the best fit of the data, and replicated signals acquired for the blank, the detection limit was about 3 ng/mL. Similar evaluation of the two additional independent calibration datasets presented in FIG. 8a resulted in detection limits of about 8 and about 3 ng/mL. This detection limit is equivalent to that obtained for a solution-based assay using a similar antibody-antigen system, yet this current platform does not suffer from the hook effect. Moreover, a filter based immunoassay using a syringe to facilitate flow rather than a vertical flow-based absorbent pad may yield a detection limit of 1-10 ng/ml for mouse IgG, but requires 1 mL of sample and 10 minutes to complete the assay rather than the 100 μL of sample and ˜2 min assay time required for the present disclosure.

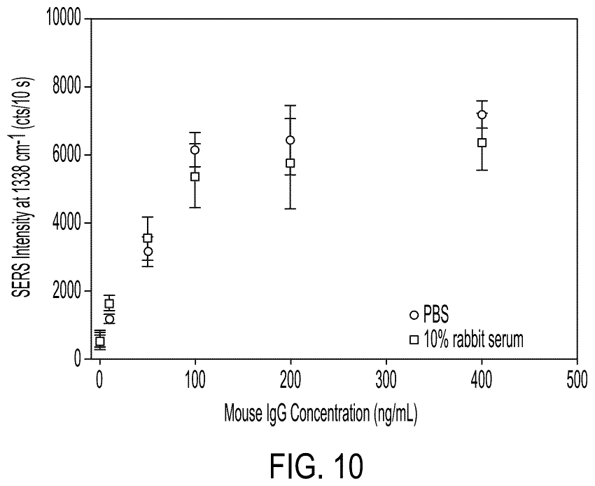

In an effort to determine the effect of sample matrix on the assay performance, calibration standard solutions of mouse IgG antigen may be prepared in PBS and in about 10% rabbit serum. The calibration curves generated with each of these solvents are overlaid in FIG. 10. Similar calibration curves are obtained for each of these sample matrices and provide two findings. First, these results confirm that the assay can capture the antigen protein in the presence of a complex biological matrix. Second, this validates the specificity of the immunoassay given that rabbit serum contains a large concentration of rabbit IgG which is not detected by the anti-mouse IgG capture substrate and ERLs.

### Application of the SERS-VFA to the Analysis of Mouse Serum

This disclosure also considers applying the SERS-VFA to determine the concentration of mouse IgG in mouse serum. IgG is an abundant protein found in serum and has normal concentration range of about 5-13 mg/mL in serum collected from healthy mice. Given that the concentration of mouse IgG in normal serum is outside of the limit of linearity for the SERS-VFA, the mouse serum may be diluted 1:10⁶ using PBS as a diluent. Three independent mouse serum samples may be prepared, and the diluted serum samples can be analyzed using a newly prepared set of plasmonic paper capture substrates. Concurrently, the capture substrates may be used to analyze mouse IgG standards to generate a calibration curve. The IgG concentration in the diluted serum measured about 11±3 ng/mL, based on the best fit of the calibration data to the ligand binding curve, which translates to an IgG concentration of about 11±3 mg/mL in the original undiluted mouse serum sample. This experimental IgG concentration in normal mouse serum is in the expected range and is consistent with measured values for mouse IgG in normal serum.

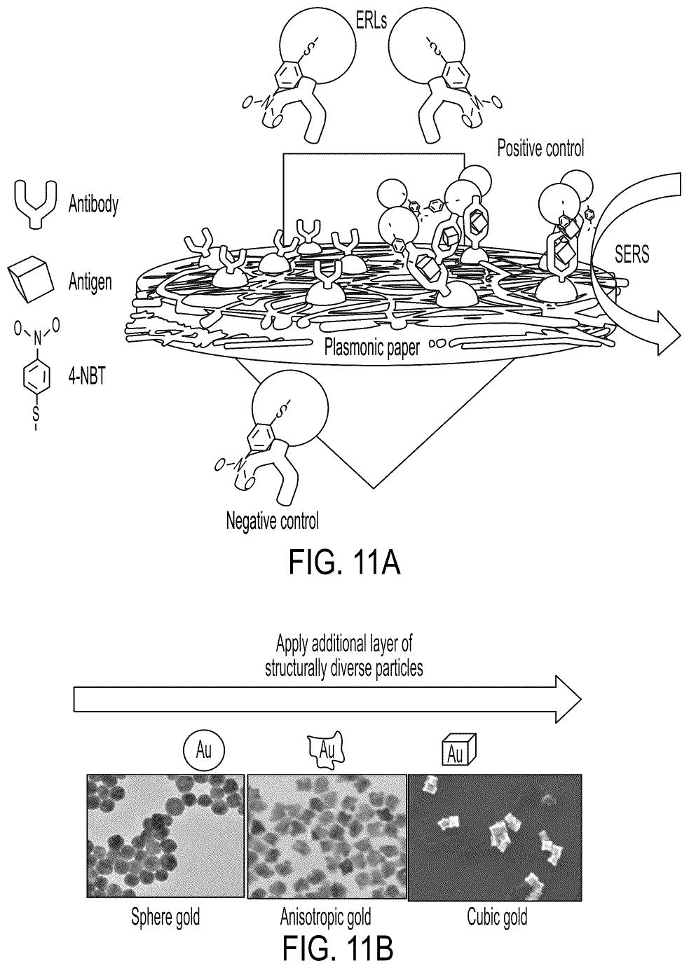

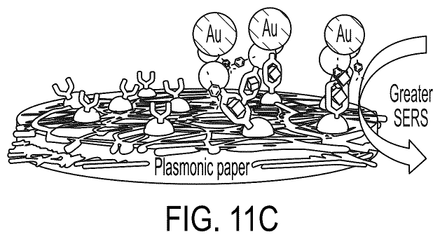

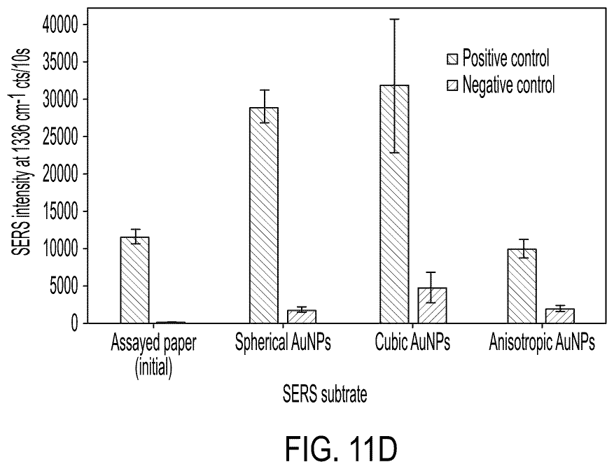

FIGS. 11a-11d illustrate a scheme for formation of third layer onto the VFA paper with structurally diverse plasmonic nanoparticles (FIG. 11a), and representative SERS signals obtained after applying spherical, anisotropic, and cubic AuNPs onto the VFA paper treated with 100 ng/ml mouse IgG (FIG. 11d).

In another aspect of this discloser a further step may be taken after the SERS-VFA (coupling between ERLs and AuNPs on filter paper). More specifically, an additional layer of structurally diverse plasmonic nanoparticles (FIG. 11b) may be used. This step provides for easily sandwiching the Raman reporter molecules (i.e., 4-NBT) that were still available around the ERLs to further increase SERS signals by creating a higher number of hot spots and SPR couplings. In one aspect of this disclosure, any one or more of three different structures of gold-based nanoparticles (e.g., sphere gold, anisotropic gold, and concave cubic gold) among others may be applied onto the assayed paper. The SERS response with the third layer may be greater (except anisotropic AuNPs) than the initial VFA, which implied additional formation of hot spots and SPR couplings. Accordingly, one embodiment of this disclosure includes introducing an additional layer with structurally diverse plasmonic nanoparticles onto the assayed paper to further improve the sensitivity of the SERS-VFA signals.

One embodiment of this disclosure utilizes a SERS-based rapid vertical flow assay employing plasmonic paper as the capture substrate. Using mouse IgG as a model antigen, this embodiment established a novel strategy to promote a significant signal enhancement for SERS detection by AuNP-embedded filter paper compared to previously utilized paper-based substrates. This disclosure takes advantage of the plasmonic coupling between the underlying plasmonic capture substrate paper and the nanoparticle labels, e.g., ERLs, to generate exceedingly large signals. Moreover, the vertical flow format enables the capture and concentration of analyte from a small volume sample for rapid detection using capillary action to actively transport the sample through the capture substrate to overcome diffusion limited mass transport. Lastly, the vertical flow approach is not complicated by the hook effect for the analysis of highly concentrated samples. The ease of use, speed of analysis, low detection limits, and low cost of components among other things suggest this assay is poised to meet the demands of point-of-care diagnostic testing, on-site environmental analysis, and point-of-need biodefense applications among other things.

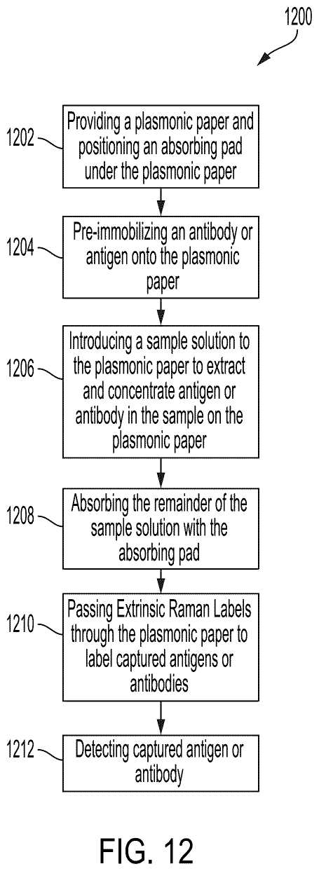

Referring now to FIG. 12, one embodiment of a diagnostic method 1200 of the present disclosure is illustrated. In box 1202, plasmonic paper as discussed herein is positioned with an absorbing pad thereunder. In box 1204 antibody (or alternatively antigen) may be pre-immobilized onto the plasmonic paper. In box 1206, a sample solution may be introduced to the plasmonic paper to extract and concentrate antigen (or alternatively antibody when antigen is pre-immobilized on the plasmonic paper) in the sample on the plasmonic paper. In box 1208, at least some of the remainder of the sample solution is absorbed with the absorbing pad. In box 1210, Extrinsic Raman Labels are passed through the plasmonic paper to label the captured antigens (or antibodies in the alternative embodiment). In box 1212, the captured antigen (or antibody in the alternative embodiment) is detected.

As noted herein, the diagnostic method 1200 may pre-immobilize antibody on the plasmonic paper to detect antigen or it may pre-immobilize antigen on the plasmonic paper to detect antibody. The particular method may vary per application and this disclosure considers pre-immobilizing either antibody or antigen on the plasmonic paper and detecting the alternative.

Another aspect of this diagnostic method may include loading the plasmonic paper with AuNP as discussed herein. In one example, the plasmonic paper is immersed in an AuNP suspension for about 24 hours before being removed and used in the diagnostic method 1200. Further, the diagnostic method 1200 may utilize plasmonic paper that is formed from grade 4 or 40 Whatman filter paper with nominal pore sizes of about 25 μm or 8 μm, respectively.

The pre-immobilizing the antibody or antigen step 1204 may utilizes about 2 μg of antibody or antigen. Further, the box 1210 may utilize about 200 μL of Extrinsic Raman Labels to pass through the plasmonic paper treated with about 100 ng/ml antigens or antibodies. The detecting box 1212 may utilize visual images of the plasmonic paper.

Another aspect of this diagnostic method 1200 includes adding an additional layer of structurally diverse plasmonic nanoparticles. In this embodiment, the additional layer comprises any one or more gold-based nanoparticles having a sphere gold, an anisotropic gold, or concave cubic gold structure. Yet another embodiment of this diagnostic method includes introducing an additional layer with plasmonic nanoparticles onto the plasmonic paper.

While an exemplary embodiment incorporating the principles of the present application has been disclosed hereinabove, the present application is not limited to the disclosed embodiments. Instead, this application is intended to cover any variations, uses, or adaptations of the application using its general principles. Further, this application is intended to cover such departures from the present disclosure as come within known or customary practice in the art to which this present application pertains and which fall within the limits of the appended claims.

The terminology used herein is for the purpose of describing particular illustrative embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations).

What is claimed is:

1. A diagnostic method, comprising:
providing a plasmonic paper and an absorbing pad positioned under the plasmonic paper;
pre-immobilizing an antibody onto the plasmonic paper;
introducing a sample solution to the plasmonic paper to extract and concentrate antigen in the sample on the plasmonic paper;
absorbing the remainder of the sample solution with the absorbing pad;
passing Extrinsic Raman Labels through the plasmonic paper to label captured antigens; and
detecting captured antigen.

2. The diagnostic method of claim 1, further wherein the plasmonic paper is AuNP-loaded.

3. The diagnostic method of claim 1, further wherein the plasmonic paper is formed from grade 4 or 40 Whatman filter paper with nominal pore sizes of about 25 μm or 8 μm, respectively.

4. The diagnostic method of claim 1, further wherein the plasmonic paper is immersed in an AuNP suspension for about 24 hours before being removed and used in the diagnostic method.

5. The diagnostic method of claim 1, further wherein the pre-immobilizing the antibody step utilizes about 2 μg of antibody.

6. The diagnostic method of claim 1, further wherein about 200 μL of Extrinsic Raman Labels are passed through the plasmonic paper treated with about 100 ng/ml antigens.

7. The diagnostic method of claim 1, further wherein the detecting step utilizes visual images of the plasmonic paper.

8. The diagnostic method of claim 1, further comprising adding an additional layer of structurally diverse plasmonic nanoparticles.

9. The diagnostic method of claim 8, further wherein the additional layer comprises any one or more gold-based nanoparticles having a sphere gold, an anisotropic gold, or concave cubic gold structure.

10. The diagnostic method of claim 1, further comprising introducing an additional layer with plasmonic nanoparticles onto the plasmonic paper.

11. A diagnostic method, comprising:
providing a plasmonic paper and an absorbing pad positioned under the plasmonic paper;
pre-immobilizing an antigen onto the plasmonic paper;
introducing a sample solution to the plasmonic paper to extract and concentrate antibodies in the sample on the plasmonic paper;
absorbing the remainder of the sample solution with the absorbing pad;
passing Extrinsic Raman Labels through the plasmonic paper to label captured antibodies; and
detecting captured antibodies.

12. The diagnostic method of claim 11, further wherein the plasmonic paper is AuNP-loaded.

13. The diagnostic method of claim 11, further wherein the plasmonic paper is formed from grade 4 or 40 Whatman filter paper with nominal pore sizes of about 25 μm or 8 μm, respectively.

14. The diagnostic method of claim 11, further wherein the plasmonic paper is immersed in an AuNP suspension for about 24 hours before being removed and used in the diagnostic method.

15. The diagnostic method of claim 11, further wherein the pre-immobilizing the antibody step utilizes about 2 μg of antibody.

16. The diagnostic method of claim 11, further wherein about 200 μL of Extrinsic Raman Labels are passed through the plasmonic paper treated with about 100 ng/ml antigens.

17. The diagnostic method of claim 11, further wherein the detecting step utilizes visual images of the plasmonic paper.

18. The diagnostic method of claim 11, further comprising adding an additional layer of structurally diverse plasmonic nanoparticles.

19. The diagnostic method of claim 18, further wherein the additional layer comprises any one or more gold-based nanoparticles having a sphere gold, an anisotropic gold, or concave cubic gold structure.

20. The diagnostic method of claim 11, further comprising introducing an additional layer with plasmonic nanoparticles onto the plasmonic paper.
